(19) United States
(12) Patent Application Publication
(10) Pub. No.: US 2007/0003510 A1
(43) Pub. Date: Jan. 4, 2007

(54) Preparations containing an extract of eperua falcata and/or constituents of the latter

(76) Inventors: Florence Henry, Villers-Les-Nancy (FR); Philippe Moser, Easey Les Nancy (FR); Louis Danoux, Saulxuree-Les-Nancy (FR); Jean-Luc Contet-Audonneau, Saint-Max (FR); Gilles Pauly, Nancy (FR)

Correspondence Address: COGNIS CORPORATION;PATENT DEPARTMENT, 300 BROOKSIDE AVENUE, AMBLER, PA, 19002, US

(21) Appl. No.: 10/547,031

(22) PCT Filed: Dec. 19, 2003
(86) PCT No.: PCT/EP03/14598

§ 371 Date: May 25, 2006

(30) Foreign Application Priority Data
Feb. 28, 2003 (EP) 03290483.1

(51) Int. Cl. A61K 9/127; A61K 8/97; A61K 36/18

(52) U.S. Cl. 424/74; 424/450; 424/725; 977/907; 977/926

(57) ABSTRACT

A cosmetic, pharmaceutical or dermatological preparation containing extract of the plant Eperua falcata, active principles of the plant Eperua falcata, astilbin or engeletin. The preparation is useful to inhibit release of pro-imflammatory mediators and neuropeptides, including CGRP and SP, for skin and hair treatment, including, sensitive skin, acne, scalp itch and neurogenous inflammation.

### FIELD OF THE INVENTION

This invention relates generally to non-therapeutic cosmetic preparations and, more particularly, to new preparations with an effective content of selected plant extracts or their active principles, to new active principles obtainable from these extracts and to the use of the extracts or active principles for the cosmetic treatment of the skin.

PRIOR ART

The human skin contains a number of nerve fibers which continuously supply the central nervous system with information on the condition of the skin and on environmental influences. These nerve fibers also include non-myelinated fibers of type C, of which the cell bodies settle in the dorsal root ganglions that are situated on both sides of the spinal column. The C fibers are sensitive to pain, are in direct contact with the cells surrounding them and influence the skin metabolism by releasing a number of messenger substances [Steinschneider et al. in Cosm'ing 2001, 2001. pp. 211-219)]. Various different neuropeptides occur in the cutanous nerve system. Switching points in which these neuropeptides are to be found concentrate themselves around the vascular plexuses and the interfaces between dermis and epidermis. In general, they correlate intensively with the cell strctures that play a key role in the development of cutaneous neurogenous inflammations, such as mast cells or keratinocytes for example.

The results of biochemical studies in recent years suggest that neuropeptides are extremely important as mediators for cutaneous neurogenous inflammations. Thus, they can be found, for example, in the same C fibers that are responsible for the reflex mechanism of the axons. Neuropeptides present in the skin are released by inflammatory stimulation. The intradermal injection of neuropeptides can release a number of typical reactions of the skin to an acute inflammation such as, for example, vasodilation, plasma extravasation, activation of mast cells or the infiltration of leucocytes [Gianetti et al. in Nouv. Dermatol. No. 16, pp. 22-23 (1997)]. In addition, inflamed regions of skin release soluble substances which activate the type C nerve fibers described at the beginning and which, in turn, again pour forth neuropeptides such as, for example, CGRP (Calcitonin gene related peptide) or SP (Substance P) [cf. Uchi et al. in J. Dermatol. Sci. 24: 529-38 (2000); Huang et al. in Neuroscience, Vol. 94, 3;965-973 (1999)]. Other neuropeptides are the substances K and P, which belong to the family of tachykines, and the substances A and B which belong to the neurokines, vasoactive intestinal peptide (VIP), peptide histidine methionine (PHM), neuropeptide Y (NPY), peptide YY (PYY), galanin, somatostatin (SO), neurotensin (NT), gastrin released peptide (GRP), bombesin, bradykinin (BK), melanostimulating hormone (MSH), serotonin (ST) or pro opiomelanocortin (POMC).

Various neuromediators are already known from the prior art. Thus, European patent EP 0723774 B1 (L'Oréal) proposes CGRP antagonists, namely CGRP 8-37 or anti-CGRP-antibodies, for non-therapeutic use in cosmetic preparations. However, these substances are mainly complex proteins which are difficult to obtain and purify and which, because of their high specialization, are only effective against a small number of the neuropeptides.

According to the present invention, astilbin was identified as a constituent of aqueous extracts of the plant Eperua falcata.

Astilbin is known per se. It is described, for example, in US 2003 016 553 3 A1, in US 2003 016 272 8 A1, in US 2003 015 258 8 A1, in US 2003 006 916 8 A1, in US 6 583 118 B1, in US 6 531 505 B2, in US 6444235 B1, in US 2002 004 005 0 A1, in US 5 650 433 A1, in EP 01 283 048 A1, in EP 1 260 517 A1, in EP 0 987 024 A2, in EP 0 742 012 A2, in EP 0 719 554 Al and in EP 0 633022 A2.

According to the invention, engeletin was identified as another constituent of aqueous extracts of the plant Eperua falcata.

Engeletin is known per se. It is described, for example, in US 2003 016 553 3 A1, in US 2003 016 272 8 A1, in US 2003 015 258 8 A1, in US 2003 0069168 A1, in US 6 583118 B1, in US 6 531 050 5 B2, in US 2002 004 005 0 A1, in US 5 650 433 A1, in EP 1 260 517 A1, in EP 0 987 024 A2, in EP 0 742 012 A2, in EP 0 719 554 A1, in EP 0 633 022 A2 and in JP 2002 173424 A.

Accordingly, the complex problem addressed by the present invention was to provide new neuromediators for the production of cosmetic preparations which would suppress the release of a broad spectrum of neuropeptides, but especially CGRP and SP. More specifically, the use of the new neuromediators would lead to cosmetic preparations which would be suitable for protecting particularly sensitive skin and which, in particular, would counteract UV-induced damage, such as, for example, vasodilation, erythema, odema and other irritations, and the release of reactive oxygen compounds (ROS=reactive oxygen species).

### DESCRIPTION OF THE INVENTION

The present invention relates to cosmetic, pharmaceutical and/or dermatological preparations which contain extracts of the plant Eperua falcata or its active principles in quantities of preferably 0.001 to 1% by weight and more particularly 0.05 to 0.1% by weight.

It has surprisingly been found that the Eperua falcata extracts satisfy the requirements stated above in excellent fashion. The extracts or the synergistic active principle mixtures present therein are readily obtainable. They inhibit both the spontaneous release and the potassium-chloride- or capsaicin-induced release of CGRP and SP. Accordingly, the substances are suitable in particular for protection against skin irritations, erythemas, inflammations and the harmful effects of UV-A and UV-B rays.

Eperua falcata

Eperua falcata (Aublet) is a tropical tree which grows to a height of 10 to 20 metres and which is found in the rain forests of Guyana, Brazil, Surinam and Venezuela. Botanically, it belongs to the family of Caesalpiniaceae and is also known by the names of wapa, bois sabre, bioudou, pangapanga, tabaca, walaba or assacu. Synonyms include Dimorpha falcata (J. B. Aublet) J. E. Smith, Panzera falcata (J. B. Aublet) C. L. Wildenow. Eperua falcata has a red-brown, very hard wood and bears dark brown, characteristically shaped fruits. The extracts, which are preferably obtained from its bark and which are used in natural medicine, for example against toothache, contain a number of active principles, more particularly polyphenols, of which some are listed by way of example in Table 1:

TABLE 1
Active principles in Eperua falcata extracts

| Active | | | Molecular | UVₘₐₓ spectrum |
|---|---|---|---|---|
| principle | Chemical affiliation | Formula | weight | (nm) alcohol |
| (+)-Catechol | Flavan-3-ols | C₁₅H₁₄O₈ | 290 | 280.5 |
| (+)-Epicatechol | Flavan-3-ols | C₁₅H₁₄O₆ | 290 | 280 |
| Procyanidin B4 | Flavan-3-ols | C₃₀H₂₆O₁₂ | 578 | 280 |
| Prodelphinidin | Flavan-3-ol gallocatechols | C₃₀H₂₆O₁₄ | 610 | 280 |
| Ellagic acid | Phenolic acids | C₁₄H₆O₈ | 302 | 256, 308 sh, 354 |
| | | | | sh, 367 |
| Eperua acid | Diterpenoids | C₂₀H₃₄O₂ | 306 | <210 |
| | Quinones | | | |

Extraction

The extracts may be prepared by methods known per se, i.e. for example by aqueous, alcoholic or aqueous/alcoholic extraction of the plants or parts thereof or the bark, leaves or fruit. Suitable extraction processes are any of the usual extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux. Percolation is advantageous for industrial use. Fresh plants or parts thereof are suitable as the starting material although dried plants and/or plant parts which may be mechanically size-reduced before extraction are normally used. Any size reduction methods known to the expert, for example freeze grinding, may be used. Preferred solvents for the extraction process are organic solvents, water (preferably hot water with a temperature above 80° C. and more particularly above 95° C.) or mixtures of organic solvents and water, more particularly low molecular weight alcohols with more or less high water contents. Extraction with methanol, ethanol, pentane, hexane, heptane, acetone, propylene glycols, polyethylene glycols, ethyl acetate and mixtures and water-containing mixtures thereof thereof is particularly preferred. The extraction process is generally carried out at 20 to 100° C., preferably at 30 to 90° C. and more particularly at 60 to 80° C. In one preferred embodiment, the extraction process is carried out in an inert gas atmosphere to avoid oxidation of the ingredients of the extract. This is particularly important where extraction is carried out at temperatures above 40° C. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried leaves are in the range from 3 to 15 and more particularly 6 to 10% by weight. The present invention includes the observation that the extraction conditions and the yields of the final extracts may be selected according to the desired application. These extracts, which generally have active substance contents (=solids contents) of 0.5 to 10% by weight, may be used as such, although the solvent may also be completely removed by drying, more particularly by spray or freeze drying, a deep red colored solid remaining behind. The extracts may also be used as starting materials for producing the pure active substances mentioned above unless they can be synthesized by a more simple and inexpensive method. Accordingly, the active substance content in the extracts may be from 5 to 100% by weight and is preferably from 50 to 95% by weight. The extracts themselves may be present as water-containing preparations and/or as preparations dissolved in organic solvents and as spray-dried or freeze-dried water-free solids. Suitable organic solvents in this connection are, for example, aliphatic alcohols containing 1 to 6 carbon atoms (for example ethanol), ketones (for example acetone), halogenated hydrocarbons (for example chloroform or methylene chloride), lower esters or polyols (for example glycerol or glycols).

Microcapsules, Liposomes and Pro-liposomes

In a preferred embodiment of the present invention, the extracts may be present in the form of microcapsules, liposomes or pro-liposomes. “Microcapsules” are understood by the expert to be spherical aggregates with a diameter of about 0.0001 to about 5 mm which contain at least one solid or liquid core surrounded by at least one continuous membrane. More precisely, they are finely dispersed liquid or solid phases coated with film-forming polymers, in the production of which the polymers are deposited onto the material to be encapsulated after emulsification and coacervation or interfacial polymerization. In another process, molten waxes are absorbed in a matrix (“microsponge”) which, as microparticles, may be additionally coated with film-forming polymers. The microscopically small capsules, also known as nanocapsules, can be dried in the same way as powders. Besides single-core microcapsules, there are also multiple-core aggregates, also known as microspheres, which contain two or more cores distributed in the continuous membrane material. In addition, single-core or multiple-core microcapsules may be surrounded by an additional second, third etc. membrane. The membrane may consist of natural, semisynthetic or synthetic materials. Natural membrane materials are, for example, gum arabic, agar agar, agarose, maltodextrins, alginic acid and salts thereof, for example sodium or calcium alginate, fats and fatty acids, cetyl alcohol, collagen, chitosan, lecithins, gelatin, albumin, shellac, polysaccharides, such as starch or dextran, polypeptides, protein hydrolyzates, sucrose and waxes. Semisynthetic membrane materials are inter alia chemically modified celluloses, more particularly cellulose esters and ethers, for example cellulose acetate, ethyl cellulose, hydroxypropyl cellulose, hydroxypropyl methyl cellulose and carboxymethyl cellulose, and starch derivatives, more particularly starch ethers and esters. Synthetic membrane materials are, for example, polymers, such as polyacrylates, polyamides, polyvinyl alcohol or polyvinyl pyrrolidone.

Examples of known microcapsules are the following commercial products (the membrane material is shown in brackets) Hallcrest Microcapsules (gelatin, gum arabic), Coletica Thalaspheres (maritime collagen), Lipotec Millicapseln (alginic acid, agar agar), Induchem Unispheres (lactose, microcrystalline cellulose, hydroxypropylmethyl cellulose), Unicerin C30 (lactose, microcrystalline cellulose, hydroxypropylmethyl cellulose), Kobo Glycospheres (modified starch, fatty acid esters, phospholipids), Softspheres (modified agar agar), Kuhs Probiol Nanospheres (phospholipids), Primaspheres and Primasponges (chitosan, alginates) and Primasys (phospholipids).

Chitosan microcapsules and processes for their production are the subject of earlier patent applications filed by applicants [WO 01/01926, WO 01/01927, WO 01/01928, WO 01/01929]. Microcapsules with mean diameters of 0.0001 to 5, preferably 0.001 to 0.5 and more particularly 0.005 to 0.1 mm, which consist of a membrane and a matrix containing the active components, may be obtained, for example, by
(a1) preparing a matrix from gel formers, chitosans and active components,
(a2) optionally dispersing the matrix in an oil phase and
(a3) treating the dispersed matrix with aqueous solutions of anionic polymers and optionally removing the oil phase in the process or
(b1) preparing a matrix from gel formers, anionic polymers and active components,
(b2) optionally dispersing the matrix in an oil phase and
(b3) treating the dispersed matrix with aqueous chitosan solutions and optionally removing the oil phase in the process or
(c1) processing aqueous active-component preparations with oil components in the presence of emulsifiers to form o/w emulsions,
(c2) treating the emulsions thus obtained with aqueous solutions of anionic polymers,
(c3) contacting the matrix thus obtained with aqueous chitosan solutions and
(c4) removing the encapsulated products thus obtained from the aqueous phase.
   Gel formers

Preferred gel formers for the purposes of the invention are substances which are capable of forming gels in aqueous solution at temperatures above 40° C. Typical examples of such gel formers are heteropolysaccharides and proteins. Preferred thermogelling heteropolysaccharides are agaroses which may be present in the form of the agar agar obtainable from red algae, even together with up to 30% by weight of non-gel-forming agaropectins. The principal constituent of agaroses are linear polysaccharides of D-galactose and 3,6-anhydro-L-galactose with alternate β-1,3- and β-1,4-glycosidic bonds. The heteropolysaccharides preferably have a molecular weight of 110,000 to 160,000 and are both odorless and tasteless. Suitable alternatives are pectins, xanthans (including xanthan gum) and mixtures thereof. Other preferred types are those which—in 1% by weight aqueous solution—still form gels that do not melt below 80° C. and solidify again above 40° C. Examples from the group of thermogelling proteins are the various gelatins.

Chitosans

Chitosans are biopolymers which belong to the group of hydrocolloids. Chemically, they are partly deacetylated chitins differing in their molecular weights which contain the following—idealized—monomer unit: 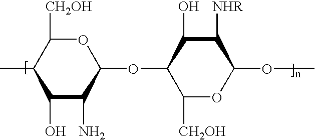
In contrast to most hydrocolloids, which are negatively charged at biological pH values, chitosans are cationic biopolymers under these conditions. The positively charged chitosans are capable of interacting with oppositely charged surfaces and are therefore used in cosmetic hair-care and body-care products and pharmaceutical preparations. Chitosans are produced from chitin, preferably from the shell residues of crustaceans which are available in large quantities as inexpensive raw materials. In a process described for the first time by Hackmann et al., the chitin is normally first deproteinized by addition of bases, demineralized by addition of mineral acids and, finally, deacetylated by addition of strong bases, the molecular weights being distributed over a broad spectrum. Preferred types are those which have an average molecular weight of 10,000 to 500,000 dalton or 800,000 to 1,200,000 dalton and/or a Brookfield viscosity (1% by weight in glycolic acid) below 5,000 mPas, a degree of deacetylation of 80 to 88% and an ash content of less than 0.3% by weight. In the interests of better solubility in water, the chitosans are generally used in the form of their salts, preferably as glycolates.
Anionic Polymers

The function of the anionic polymers is to form membranes with the chitosans. Preferred anionic polymers are salts of alginic acid. The alginic acid is a mixture of carboxyl-containing polysaccharides with the following idealized monomer unit:

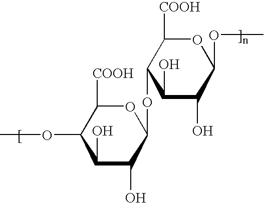

The average molecular weight of the alginic acid or the alginates is in the range from 150,000 to 250,000. Salts of alginic acid and complete and partial neutralization products thereof are understood in particular to be the alkali metal salts, preferably sodium alginate (“algin”), and the ammonium and alkaline earth metal salts. Mixed alginates, for example sodium/magnesium or sodium/calcium alginates, are particularly preferred. In an alternative embodiment of the invention, however, anionic chitosan derivatives, for example carboxylation and above all succinylation products are also suitable for this purpose. Alternatively, poly(meth)acrylates with average molecular weights of 5,000 to 50,000 dalton and the various carboxy-methyl celluloses may also be used. Instead of the anionic polymers, anionic surfactants or low molecular weight inorganic salts, such as pyrophosphates for example, may also be used for forming the membrane.

Production Process for Microcapsules

To produce the microcapsules, a 1 to 10 and preferably 2 to 5% weight aqueous solution of the gel former, preferably agar agar, is normally prepared and heated under reflux. A second aqueous solution containing the chitosan in quantities of 0.1 to 2 and preferably 0.25 to 0.5% by weight and the active substances in quantities of 0.1 to 25 and preferably 0.25 to 10% by weight is added in the boiling heat, preferably at 80 to 100° C.; this mixture is called the matrix. Accordingly, the charging of the microcapsules with active substances may also comprise 0.1 to 25% by weight, based on the weight of the capsules. If desired, water-insoluble constituents, for example inorganic pigments, may be added at this stage to adjust viscosity, generally in the form of aqueous or aqueous/alcoholic dispersions. In addition, to emulsify or disperse the active substances, it can be useful to add emulsifiers and/or solubilizers to the matrix. After its preparation from gel former, chitosan and active substances, the matrix may optionally be very finely dispersed in an oil phase with intensive shearing in order to produce small particles in the subsequent encapsulation process. It has proved to be particularly advantageous in this regard to heat the matrix to temperatures in the range from 40 to 60° C. while the oil phase is cooled to 10 to 20° C. The actual encapsulation, i.e. formation of the membrane by contacting the chitosan in the matrix with the anionic polymers, takes place in the last, again compulsory step. To this end, it is advisable to wash the matrix optionally dispersed in the oil phase with an aqueous ca. 1 to 50 and preferably 10 to 15% by weight aqueous solution of the anionic polymer at a temperature of 40 to 100° C. and preferably at a temperature of 50 to 60° C. and, if necessary, to remove the oil phase either at the same time or afterwards. The resulting aqueous preparations generally have a microcapsule content of 1 to 10% by weight. In some cases, it can be of advantage for the solution of the polymers to contain other ingredients, for example emulsifiers or preservatives. After filtration, microcapsules with a mean diameter of preferably about 1 mm are obtained. It is advisable to sieve the capsules to ensure a uniform size distribution. The microcapsules thus obtained may have any shape within production-related limits, but are preferably substantially spherical. Alternatively, the anionic polymers may also be used for the preparation of the matrix and encapsulation may be carried out with the chitosans.

An alternative process for the production of the microcapsules according to the invention comprises initially preparing an o/w emulsion which, besides the oil component, water and the active components, contains an effective quantity of emulsifier. To form the matrix, a suitable quantity of an aqueous anionic polymer solution is added to this preparation with vigorous stirring. The membrane is formed by addition of the chitosan solution. The entire process preferably takes place at a mildly acidic pH of 3 to 4. If necessary, the pH is adjusted by addition of mineral acid. After formation of the membrane, the pH is increased to a value of 5 to 6, for example by addition of triethanolamine or another base. This results in an increase in viscosity which can be supported by addition of other thickeners such as, for example, polysaccharides, more particularly xanthan gum, guar guar, agar agar, alginates and tyloses, carboxymethyl cellulose and hydroxyethyl cellulose, relatively high molecular weight polyethylene glycol mono- and diesters of fatty acids, polyacrylates, polyacrylamides and the like. Finally, the microcapsules are separated from the aqueous phase, for example by decantation, filtration or centrifuging.

Liposomes and Pro-liposomes

Instead of the described microcapsules, pro-liposomes may also be used as carriers for the active-principle mixtures. By way of explanation, it is pointed out that pro-liposomes do not contain any water and only absorb water to form true liposomes when they are introduced into a watery environment. Pro-liposomes in the context of the invention can be obtained by treating the active principles with lecithins and/or phospholipids in cosmetically or pharmaceutically acceptable solvents. To this end, the active principles are normally either introduced into a solvent and contacted with lecithins or phospholipids at temperatures of 30 to 70° C. or the water-free mixtures are stirred into a solution of the lecithins or phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs) Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Nanoparticles

In another preferred embodiment of the invention, the extracts may be present as fine-particle solids with a mean particle diameter in the range from 100 to 300, preferably 50 to 200 and more particularly 100 to 150 nm (“nanoparticles”). These nanoparticles are normally produced by the evaporation technique which has parallels with conventional spray drying. In the evaporation technique, the starting materials are first dissolved in a suitable organic solvent (for example alkanes, vegetable oils, ethers, esters, ketones, acetals and the like). The resulting solutions are then introduced into water or another nonsolvent—optionally in the presence of a surface-active compound dissolved therein—so that the homogenization of the two immiscible solvents results in precipitation of the nanoparticles, the organic solvent preferably evaporating. O/w emulsions or o/w micro-emulsions may be used instead of an aqueous solution. Instead of the evaporation technique, other known processes may be used, including, for example:

RESS Process

In the RESS process, nanoparticles are produced by the rapid expansion of supercritical solutions. This process leads to particularly homogeneous distribution of the particle diameters. To this end, the extracts are preferably dissolved in a suitable solvent under supercritical or near-critical conditions, the fluid mixture is expanded through a nozzle into a vacuum, a gas or a liquid and the solvent is simultaneously evaporated. To prevent the nanoparticles from agglomerating, it is advisable to dissolve the starting materials in the presence of suitable protective colloids or emulsifiers and/or to expand the critical solutions into aqueous and/or alcoholic solutions of the protective colloids or emulsifiers or into cosmetic oils which may in turn contain redissolved emulsifiers and/or protective colloids. Suitable protective colloids are, for example, gelatine, casein, gum arabic, lysalbinic acid, starch and polymers, such as polyvinyl alcohols, polyvinyl pyrrolidones, polyalkylene glycols and polyacrylates.

GAS, PCA or PGSS Process

Another method for the production of nanoparticles is the so-called GAS process (gas anti-solvent recrystallization). This process uses a highly compressed gas or supercritical fluid (for example carbon dioxide) as non-solvent for the crystallization of dissolved substances. The compressed gas phase is introduced into the primary solution of the starting materials and absorbed therein so that there is an increase in the liquid volume and a reduction in solubility and fine particles are precipitated.

The PCA process (precipitation with a compressed fluid anti-solvent) is equally suitable. In this process, the primary solution of the starting materials is introduced into a supercritical fluid which results in the formation of very fine droplets in which diffusion processes take place so that very fine particles are precipitated.

In the PGSS process (particles from gas saturated solutions), the starting materials are melted by the introduction of gas under pressure (for example carbon dioxide or propane). Temperature and pressure reach near- or super-critical conditions. The gas phase dissolves in the solid and lowers the melting temperature, the viscosity and the surface tension. On expansion through a nozzle, very fine particles are formed as a result of cooling effects.

Commercial Applications

The present invention also relates to the use of extracts of the plant Eperua falcata or its active principles for the production of non-therapeutic cosmetic, pharmaceutical and/or dermatological preparations, more particularly skin and hair treatment preparations, in which they may be present in quantities of preferably 0.001 to 5% by weight and more particularly 0.05 to 1% by weight.

The present invention also relates to the use of extracts of the plant Eperua falcata or its active principles
for inhibiting neuropeptides or for reducing the release of pro-inflammatory mediators of stressed skin cells or neuronal fibers, the neuropeptides or mediators preferably being CGRP or SP;
for reducing neurogenous inflammations;
for treating sensitive skin;
as anti-acne agents and
for reducing scalp itch.
   Cosmetic, Pharmaceutical and/or Dermatological Preparations

The extracts according to the invention may be used for the production of cosmetic, pharmaceutical or dermatological preparations such as, for example, hair shampoos, hair lotions, foam baths, shower baths, creams, gels, lotions, alcoholic and aqueous/alcoholic solutions, emulsions, wax/fat compounds, stick preparations, powders or ointments. These preparations may also contain mild surfactants, oil components, emulsifiers, pearlizing waxes, consistency factors, thickeners, superfatting agents, stabilizers, polymers, silicone compounds, fats, waxes, lecithins, phospholipids, UV protection factors, biogenic agents, antioxidants, deodorizers, antiperspirants, antidandruff agents, film formers, swelling agents, insect repellents, self-tanning agents, tyrosine inhibitors (depigmenting agents), hydrotropes, solubilizers, preservatives, perfume oils, dyes and the like as further auxiliaries and additives.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, alkyl ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkyl hydroxycarboxylic acids with linear or branched C₆₋₂₂ fatty alcohols (cf. DE 19756377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-/di-/triglyceride mixtures based on C₆₋₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, for example Dicaprylyl Carbonate (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, for example Dicaprylyl Ether (Cetiol® OE), ring opening products of epoxidized fatty acid esters with polyols, silicone oils (cyclomethicone, silicon methicones, etc.) and/or aliphatic or naphthenic hydrocarbons such as, for example, squalane, squalene or dialkyl cyclohexanes.

Emulsifiers

Suitable emulsifiers are, for example, nonionic surfactants from at least one of the following groups:
products of the addition of 2 to 30 mol ethylene oxide and/or 0 to 5 mol propylene oxide onto linear C₈₋₂₂ fatty alcohols, C₁₂₋₂₂ fatty acids, alkyl phenols containing 8 to 15 carbon atoms in the alkyl group and alkylamines containing 8 to 22 carbon atoms in the alkyl group;
alkyl and/or alkenyl oligoglycosides containing 8 to 22 carbon atoms in the alk(en)yl group and ethoxylated analogs thereof;
products of the addition of 1 to 15 mol ethylene oxide onto castor oil and/or hydrogenated castor oil;
products of the addition of 15 to 60 mol ethylene oxide onto castor oil and/or hydrogenated castor oil;
partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof with 1 to 30 mol ethylene oxide;
partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof with 1 to 30 mol ethylene oxide;
mixed esters of pentaerythritol, fatty acids, citric acid and fatty alcohol and/or mixed esters of fatty acids containing 6 to 22 carbon atoms, methyl glucose and polyols, preferably glycerol or polyglycerol;
mono-, di- and trialkyl phosphates and mono-, di- and/or tri-PEG-alkyl phosphates and salts thereof;
wool wax alcohols;
polysiloxane/polyalkyl/polyether copolymers and corresponding derivatives;
block copolymers, for example Polyethyleneglycol-30 Dipolyhydroxystearate;
polymer emulsifiers, for example Pemulen types (TR-1, TR-2) from Goodrich;
polyalkylene glycols and
glycerol carbonate.
   Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out.

C_{21/18} fatty acid monoesters and diesters of addition products of ethylene oxide onto glycerol are known as lipid layer enhancers for cosmetic formulations.

Alkyl and/or Alkenyl Oligoglycosides

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may also contain small quantities of triglyceride from the production process. Products of the addition of I to 30 and preferably 5 to 10 mol ethylene oxide onto the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers

Typical anionic emulsifiers are aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes, microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, also relatively high molecular weight polyethylene glycol monoesters and diesters of fatty acids, polyacrylates (Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryidimonium Hydroxypropyl Hydrolyzed Collagen (Lamequat® L, Grünau), quaternized wheat poly-peptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), the polyaminopolyamides described, for example, in FR 2252840 A and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

UV Protection Factors

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. The UV protection factors are present in quantities of normally 0.1 to 5% by weight and preferably 0.2 to 1% by weight. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor;
4-aminobenzoic acid derivatives, preferably 4-(dimethylamino)-benzoic acid-2-ethylhexyl ester, 4-(dimethylamino)-benzoic acid-2-octyl ester and 4-(dimethylaminoy)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789), 2-(4-diethylamino-2-hydroxybenzoyl)-benzoic acid hexyl ester (Uvinu® A Plus), 1-phenyl-3-(4′-isopropylphenyl)-propane-1,3-dione and enamine compounds. The UV-A and UV-B filters may of course also be used in the form of mixtures. Particularly favorable combinations consist of the derivatives of benzoyl methane, for example 4-tert.butyl4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000. Suitable hydrophobic coating materials are, above all, silicones and, among these, especially trialkoxyoctylsilanes or simethicones. So-called micro- or nanopigments are preferably used in sun protection products. Micronized zinc oxide is preferably used.

Biogenic Agents and Antioxidants

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes.

Antioxidants interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmol to μmol/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxybenzoic acid and salts and esters thereof, N-(4-chloro-phenyl)-N′-(3,4-dichlorophenyl)-urea, 2,4,4′-trichloro-2′-hydroxy-diphenylether (triclosan), 4-chloro-3,5-dimethylphenol, 2,2′-methylene-bis-(6-bromo-4-chlorophenol), 3-methyl-4-(1-methyl-ethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, a-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:
astringent active principles,
oil components,
nonionic emulsifiers,
co-emulsifiers,
consistency factors,
auxiliaries in the form of, for example, thickeners or complexing agents and/or
non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,
inflammation-inhibiting, skin-protecting or pleasant-smelling essential oils,
synthetic skin-protecting agents and/or
oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Piroctone Olamine (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Self-tanning and Depigmenting Agents

A suitable self-tanning agent is dihydroxyacetone. Suitable tyrosine inhibitors, which prevent the formation of melanin and are used in depigmenting preparations, are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.
   Preservatives

Suitable preservatives are, for example, phenoxyethanol, formaldehyde solution, parabens, pentanediol or sorbic acid, the silver complexes known by the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, a-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Fäirbemittel” of the Farbstoffkommission der Deutschen Forschungsgemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparation. The preparations may be produced by standard hot or cold processes and are preferably produced by the phase inversion temperature method.

### EXAMPLES

5.3 kg distilled water were introduced into a reactor and 600 g size-reduced Eperua falcata bark were added. The mixture was then heated for 1 hour to 85° C., cooled to 20° C. and centrifuged for 15 mins. to remove insoluble components. 300 g dextrin were then added and the solution was passed through a 45 μm mesh filter. Finally, the solution obtained was spray-dried to form a dry powder containing 10% by weight extract and 90% by weight dextrin. The yield amounted to ca. 14% by weight, based on the plant material used.

Cell Toxicity and Inhibition of Neuropeptides

The inhibiting effect of the extracts according to the invention was tested in vitro on a neurone culture which had been incubated for 2 weeks at 37° C./5% by volume carbon dioxide. After the culture medium had been replaced, treatment with the Eperua extract prepared as described above was carried out. The neurones were then stimulated either by addition of a preparation which depolarized the cell membranes of the neurones (40 mM potassium chloride) or by addition of 10 μM capsaicin (which is known to form erythemas) over a period of 20 mins. at 37° C./5% by volume carbon dioxide.

In a first series of tests, the cell toxicity of the test substances used was determined via the quantity of lactate dehydrogenase (LDH) released using Bonnekoh's method (cf. Dermatol. Res. 22, pp. 325-329 (1990)]. The quantity of neuropeptides released as a result of the stimulation was then measured by the ELISA method. The results are set out in Table 2. The LDH and CGRP values measured are based on the standard (=no addition of a test substance).

TABLE 2
Cell toxicity and inhibition of neuropeptides

| Active principle | Concentration | LDH | CGRP |
|---|---|---|---|
| None | — | 100 ± 0 | 100 ± 21 |
| Eperua extract | 0.005% w/v | 82 ± 6 | |
| Eperua extract | 0.05% w/v | 83 ± 6 | |
| KCl | 40 mM | | 554 ± 8 |
| Eperua extract | 0.01% w/v | | 0 |
| Eperua extract + KCl | 0.01% w/v + 40 mM | | 233 ± 40 |
| Capsaicin | 10 μM | | 520 ± 107 |
| Eperua extract + | 0.01% w/v + 10 μM | | 293 ± 118 |
| capsaicin | | | |

Discussion of Results
It can be seen from the quantities of LDH released that the extracts have no toxic effects on the neurones, even in different concentrations.
It can be seen from the quantity of CGRP released that the addition of KCl or capsaicin leads to intensive stimulation which can be significantly reduced by addition of the extracts according to the invention.

Table 3 contains a number of Formulation Examples.

TABLE 3
Examples for cosmetic preparations (water, preservative to 100% by wt.)

| Composition (INCI) | 1 | 2 | 3 | 4 | 5 | 6 | 7 | 8 | 9 | 10 |
|---|---|---|---|---|---|---|---|---|---|---|
| Dehymuls ® PGPH | 4.0 | 3.0 | — | 5.0 | — | — | — | — | — | — |
| Polyglyceryl-2 Dipolyhydroxystearate | | | | | | | | | | |
| Lameform ® TGI | 2.0 | 1.0 | — | — | — | — | — | — | — | — |
| Polyglyceryl-3 Diisostearate | | | | | | | | | | |
| Emulgade ® PL 68/50 | — | — | — | — | 4.0 | — | — | — | 3.0 | — |
| Cetearyl Glucoside (and) Cetearyl Alcohol | | | | | | | | | | |
| Eumulgin ® B2 | — | — | — | — | — | — | — | 2.0 | — | — |
| Ceteareth-20 | | | | | | | | | | |
| Tegocare ® PS | — | — | 3.0 | — | — | — | 4.0 | — | — | — |
| Polyglyceryl-3 Methylglucose Distearate | | | | | | | | | | |
| Eumulgin ® VL 75 | — | — | — | — | — | 3.5 | — | — | 2.5 | — |
| Polyglyceryl-2 Dipolyhydroxystearate (and) Lauryl | | | | | | | | | | |
| Glucoside (and) Glycerin | | | | | | | | | | |
| Bees Wax | 3.0 | 2.0 | 5.0 | 2.0 | — | — | — | — | — | — |
| Cutina ® GMS | — | — | — | — | — | 2.0 | 4.0 | — | — | 4.0 |
| Glyceryl Stearate | | | | | | | | | | |
| Lanette ® O | — | — | 2.0 | — | 2.0 | 4.0 | 2.0 | 4.0 | 4.0 | 1.0 |
| Cetearyl Alcohol | | | | | | | | | | |
| Antaron ® V 216 | — | — | — | — | — | 3.0 | — | — | — | 2.0 |
| PVP/Hexadecene Copolymer | | | | | | | | | | |
| Myritol ® 818 | 5.0 | — | 10.0 | — | 8.0 | 6.0 | 6.0 | — | 5.0 | 5.0 |
| Cocoglycerides | | | | | | | | | | |
| Finsolv ® TN | — | 6.0 | — | 2.0 | — | — | 3.0 | — | — | 2.0 |
| C12/15 Alkyl Benzoate | | | | | | | | | | |
| Cetiol ® J 600 | 7.0 | 4.0 | 3.0 | 5.0 | 4.0 | 3.0 | 3.0 | — | 5.0 | 4.0 |
| Oleyl Erucate | | | | | | | | | | |
| Cetiol ® OE | 3.0 | — | 6.0 | 8.0 | 6.0 | 5.0 | 4.0 | 3.0 | 4.0 | 6.0 |
| Dicaprylyl Ether | | | | | | | | | | |
| Mineral Oil | — | 4.0 | — | 4.0 | — | 2.0 | — | 1.0 | — | — |
| Cetiol ® PGL | — | 7.0 | 3.0 | 7.0 | 4.0 | — | — | — | 1.0 | — |
| Hexadecanol (and) Hexyldecyl Laurate | | | | | | | | | | |
| Bisabolol | 1.2 | 1.2 | 1.2 | 1.2 | 1.2 | 1.2 | 1.2 | 1.2 | 1.2 | 1.2 |
| Eperua extract | 0.05 | 0.05 | 0.05 | 0.05 | 0.05 | 0.05 | 0.05 | 0.05 | 0.05 | 0.05 |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Copherol ® F 1300 | 0.5 | 1.0 | 1.0 | 2.0 | 1.0 | 1.0 | 1.0 | 2.0 | 0.5 | 2.0 |
| Tocopherol/Tocopheyl Acetate | | | | | | | | | | |
| Neo Heliopan ® Hydro | 3.0 | — | — | 3.0 | — | — | 2.0 | — | 2.0 | — |
| Sodium Phenylbenzimidazole Sulfonate | | | | | | | | | | |
| Neo Heliopan ® 303 | — | 5.0 | — | — | — | 4.0 | 5.0 | — | — | 10.0 |
| Octocrylene | | | | | | | | | | |
| Neo Heliopan ® BB | 1.5 | — | — | 2.0 | 1.5 | — | — | — | 2.0 | — |
| Benzophenone-3 | | | | | | | | | | |
| Neo Heliopan ® E 1000 | 5.0 | — | 4.0 | — | 2.0 | 2.0 | 4.0 | 10.0 | — | — |
| Isoamyl p-Methoxycinnamate | | | | | | | | | | |
| Neo Heliopan ® AV | 4.0 | — | 4.0 | 3.0 | 2.0 | 3.0 | 4.0 | — | 10.0 | 2.0 |
| Octyl Methoxycinnamate | | | | | | | | | | |
| Uvinul ® T 150 | 2.0 | 4.0 | 3.0 | 1.0 | 1.0 | 1.0 | 4.0 | 3.0 | 3.0 | 3.0 |
| Octyl Triazone | | | | | | | | | | |
| Zinc Oxide | — | 6.0 | 6.0 | — | 4.0 | — | — | — | — | 5.0 |
| Titanium Dioxide | — | — | — | — | — | — | — | 5.0 | — | — |
| Glycerin (86% by wt.) | 5.0 | 5.0 | 5.0 | 5.0 | 5.0 | 5.0 | 5.0 | 5.0 | 5.0 | 5.0 |

(1) W/O sun protection cream, (2-4) W/O sun protection lotion, (5, 8, 10) O/W sun protection lotion, (6, 7, 9) O/W sun protection cream

Identification of New Polyphenols

In all 15 new compounds (A to O in the following) were isolated from the aqueous Eperua extract prepared in accordance with H1 (but without addition of dextrin). These new compounds were polyphenols, preferably of the flavan and chalcone type, and were isolated by HPLC (HP 1100, Photo Diode Array Detector, HP Software; Hewlett-Packard). Separation was carried out with aqueous methanol, a concentration gradient being applied, namely 0 to 35 mins. H₂O/MeOH=70:30, 35 to 37 mins.=20:80, then change to pure methanol. The quantity injected amounted to 10 μl. UV detection was carried out at 210, 254 and 363 nm. The column (SymmetryShield® RP-18 (250×4.6, Waters) was operated at 25° C. The LC/MS-MS analysis was carried out under the following operating conditions (Table 4):

TABLE 4
LC/MS-MS process conditions

| Parameter | Value |
|---|---|
| Capillary temperature | 150° C. |
| Nebulizer gas | 60 psi |
| Vaporization temperature | 450° C. |
| Voltage Source | 6.0 kV |
| Current Source | 4.5 μA |
| Mass range | 150-800 Da |
| Analytical temperature | 35 min |
| Source | APCI |
| Mode | Positive |
| Collision energy | 40% |

Table 5 below shows the retention times, basic ions and UV absorption with which the new compounds can be identified.

TABLE 5
LC/MS-MS analysis of compounds A to O

| Retention | | | | | UVₘₐₓ |
|---|---|---|---|---|---|
| time | | | | | Spectrum |
| [min] | No. | Basic ion | Positive APCI | Elimination | [nm] |
| 16.70 | A | 341 | 323 > 305 > 287 > 261 > 190 | H₂O > H₂O > H₂O | 196 |
| 18.11 | B | 451 | 467 > 451 > 433 > 415 > 397 > 387 > 371 > | O > H₂O > H₂O > H₂O > H₂O > H₂O > H₂O > CO | 200-294 |
| | | | 353 > 347 > 329 > 299 > 263 > 245 > 219 > 191 | | |
| 18.73 | C | 451 | 467 > 451 > 433 > 415 > 397 > 387 > 371 > | O > H₂O > H₂O > H₂O > H₂O > H₂O > H₂O | 200-294 |
| | | | 353 > 347 > 329 > 299 > 263 | | |
| 19.33 | D | 451 | 467 > 451 > 433 > 415 > 397 > 387 > 371 > | O > H₂O > H₂O > H₂O > H₂O > H₂O > H₂O | 200-294 |
| | | | 353 > 347 > 329 > 299 > 263 | | |
| 19.93 | E | 451 | 467 > 451 > 433 > 415 > 397 > 387 > | O > H₂O > H₂O > H₂O > H₂O > H₂O > H₂O | 200-294 |
| | | | 371 > 353 > 219 > 191 | | |
| 20.57 | F | 435 | 451 > 435 > 399 > 381 > 353 > 33 | O > H₂O > H₂O > H₂O > CO > H₂O | 200-217-294 |
| | | | 31 > 309 > 285 > 263 > 245 > 219 > 215 | | |
| 21.85 | G | 435 | 451 > 435 > 399 > 381 > 353 > 33 | O > H₂O > H₂O > H₂O > CO > H₂O | 200-217-294 |
| | | | 31 > 309 > 285 > 263 > 245 | | |
| 23.24 | H | 435 | 451 > 435 > 399 > 381 > 353 > 33 | O > H₂O > H₂O > H₂O > CO > H₂O | |
| | | | 31 > 309 > 285 > 263 > 245 > 219 > 215 > 195 | | |
| 10.51 | I | 437 | 419 > 401 > 383 > 357 > | H₂O > H₂O > H₂O > H₂O > Prenyl | 196 |
| | | | 315 > 285 > 267 > 231 > 163 | | |
| 24.01 | J | — | 570 > 432 > 287 > 267 | | 196 |
| 26.48 | K | 287 | 570 > 432 > 287 > 267 | | 196 |
| 27.10 | L | 287 | 570 > 432 > 287 > 267 | | 196 |
| 29.16 | M | 287 | 570 > 432 > 287 > 267 | | 196 |
| 30.21 | N | 287 | 570 > 432 > 287 > 267 | | 196 |
| 30.48 | O | 287 | 570 > 432 > 287 > 267 | | 196 |

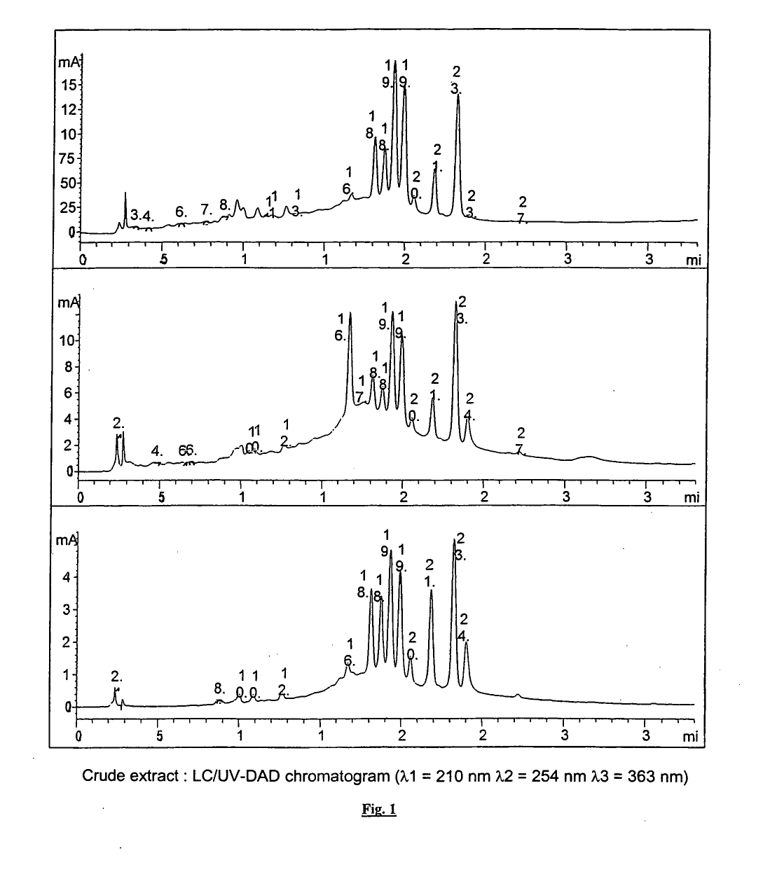

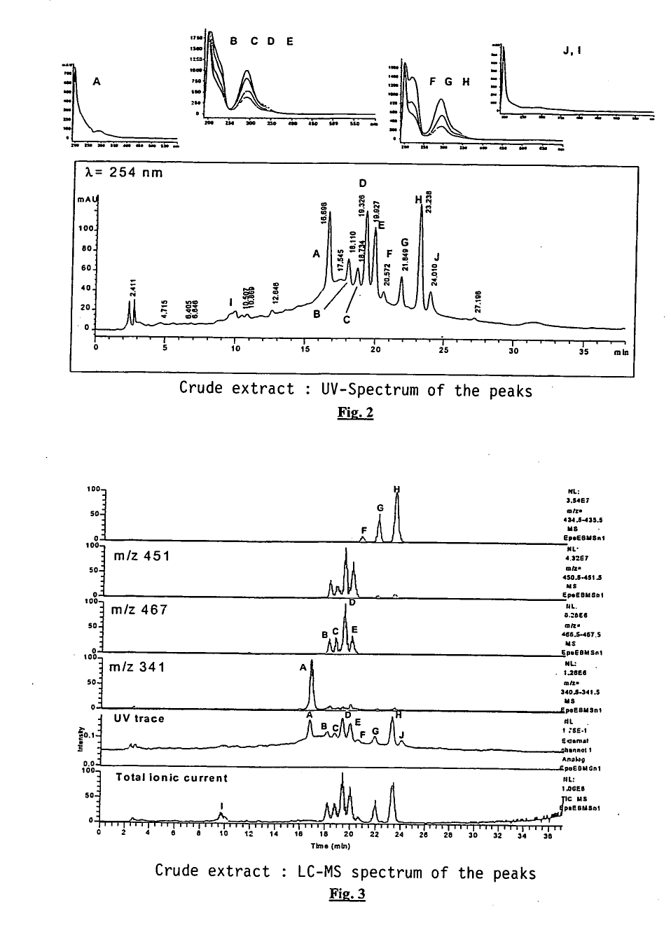

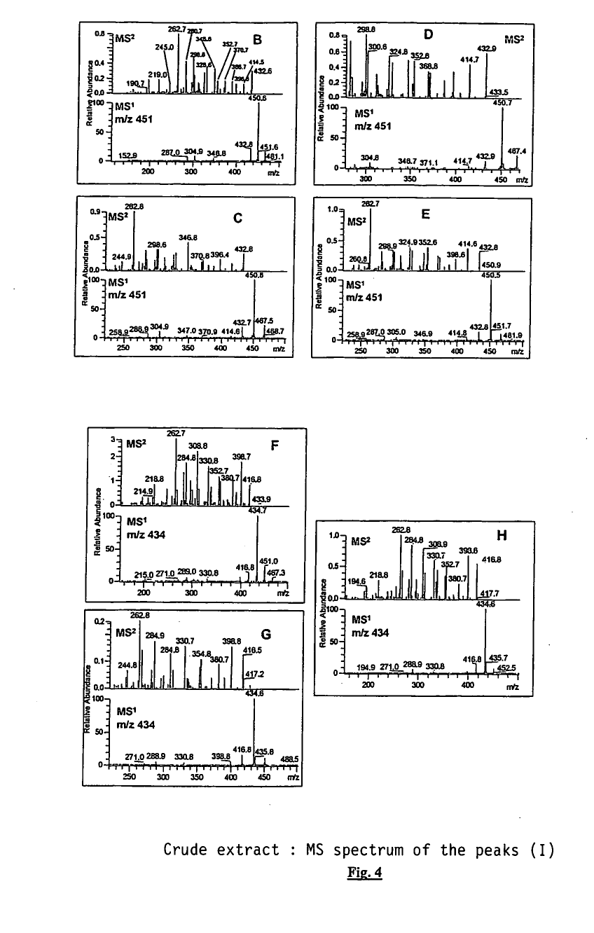

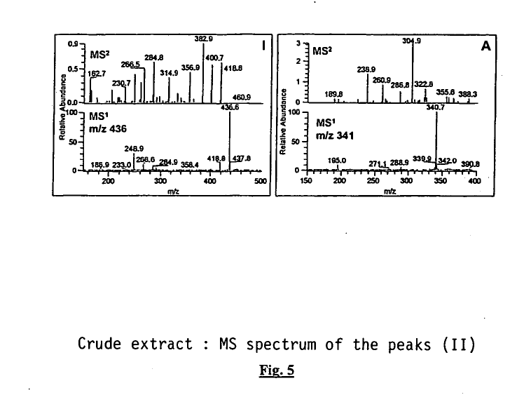

FIGS. 1 to 5 show the corresponding chromatograms and spectra for the 15 new compounds A to O.

Compounds D and H in Table 5 represent the largest peaks in the chromatograms. These two compounds, which both belong to the class of dihydroflavonols, were identified, in particular using ^{1H}HNMR and ^{13C}NMR.

Dihydroflavonols may also be referred to as 3-hydroxyflavonones or as flavonone-3-ols. Biosynthetically, they are formed by oxidation at the C-3 of the flavanones. Dihydroflavonols have two chiral centers at C-2 and at C-3. Most naturally occurring dihydroflavonols are stereochemically present in the 2R,R form.

Compound D is astilbin. Astilbin has the Chemical Abstracts Registration No. 29838-67-3. Synonyms for astilbin are 3,3′, 4′, 5,7-pentahydroxyflavanone, 3-O-L-rhamnopyranside, dihydroquercetin, 3-O-L-rhamnopyranoside. The empirical formula for astilbin is C₂₁H₂₂O₁₁; its molecular weight is 450,398 g/mol. Astilbin occurs naturally in Taxillus kaempferi, Vitis vinifera, Encryphia cordifolia, Astilbe, Quintinia, Litsea spp.

Compound H is engeletin. Engeletin has the Chemical Abstracts Registration No. 572-31-6. Synonyms for engeletin are 3,4′, 5,7-tetrahydroxyflavanone, 3-O-L-rhamnopyranoside, dihydrokaempferol-3-rhamnoside. The empirical formula for engeletin is C₂₁H₂₂O₁₀; its molecular weight if 434.399 g/mol. Engeletin occurs naturally in Engelhardtia, Eucalyptus, Eucryphia, Flindersia, Glycoxylon, Lyonia, Nothofagus, Smilax and Vitis spp.

Properties of Astilbin and Engeletin: Anti-Inflammatory Effect in vitro: Ex. A:

The protective effect of astilbin and engeletin against UV-B radiation on human keratinocytes is demonstrated in the following.

UV-B radiation (wavelength 280 to 320 nm) causes inflammation of the skin, mainly by activation of an enzyme (phospholipase A2 or PLA2) which releases arachidonic acid from the cell membrane (V. A. De Leo, D. Hanson, I. B. Weinstein and L. C. Harber, Ultraviolet radiation stimulates the release of arachidonic acid from mammalian cells in culture, Photochemistry and Photobiology (1985), Vol. 41, No. 1, pages 51 to 56). Other enzymes (so-called cyclo-oxygenases) then convert the arachidonic acid into active components, so-called prostaglandin (PG for short). The prostaglandin is secreted by the cells. The fixing of certain prostaglandins (PGE2) to specific receptors of the skin leads to reddening and swelling of the skin in the same way as sunburn does.

In cultured human cells, these effects of UV-B radiation on the cell membranes are associated with the release of a cytoplasmatic enzyme into the supernatant solution. This cytoplasmatic enzyme is lactate dehydrogenase or LDH (B. Bonnekoh, B. Farkas, J. Geisel and G. Mahrle, Lactate dehydrogenase release as an indicator of dithranol-induced membrane injury in cultured human keratinocytes, Dermatological Research (1990), Vol. 282, pages 325-331).

The experiment was carried out as follows:

Human keratinocytes were incubated for three days at 37° C. in an atmosphere containing 5% by volume carbon dioxide (standard nutrient medium containing foetal calf serum (FCS)). The nutrient medium was then replaced by an isotonic common salt solution containing the substance to be tested (astilbin or engeletin). The keratinocytes were exposed to UV-B radiation (50 mJ/cm², DUKE GL40E lamp), followed by incubation for one day at 37° C. in an atmosphere containing 5% by volume carbon dioxide. The supernatant nutrient medium was spectroscopically analyzed for LDH2 and PEG2 by the ELISA method.

The results are set out in the following Table (% by comparison with the control test).

| | Quantity of LDH | Quantity of PGE2 |
|---|---|---|
| | in %/(control | in %/(control |
| | without UV-B | without UV-B |
| | exposure) | exposure) |
| Control without UV-B | 0 | 0 |
| exposure | | |
| UV-B 50 mJ/cm² | 100 | 100 |
| Pure engeletin: 0.003% | 47 | 35 |
| Pure astilbin: 0.003% | 58 | 58 |

These results show that 0.003% astilbin reduced the quantity of LDH and PGE2 by comparison with the control test. This reflects an anti-inflammatory effect. The same applies to engeletin although the effect is not as pronounced. Accordingly, the two substances astilbin and engeletin contribute significantly to the properties of the extract of the plant Eperua falcata.

Properties of Astilbin and Engeletin: Anti-Inflammatory Effect in vitro: Ex. B:

In the course of cutaneous inflammation, leucocytes, for example polymorphonuclear neutrophilic granulocytes (PMNs), are attracted and stimulated by peptides, such as cytokines and other messenger substances such as, for example, leudotrienes released from activated or necrotic cells in the dermis. These stimulated PMNs secrete not only pro-inflammatory cytokines, leucotrienes and proteases, but also reactive oxygen compounds (ROS), such as superoxides and hypochlorite anions for example. This is done to destroy pathogenic bacteria or fungi. This activity of the PMNs is known as respiratory burst and intensifies the inflammation reaction and causes tissue damage through released ROS and lysosomal enzymes.

Particulars of the test procedure can be found in the article by Kapp et al. entitled “The role of immunomodulating cytokines, activation of the oxidative metabolism in human polymorphonuclear neutrophilic granulocytes, Journal of Investigative Dermatology, Vol. 95, pages 94S to 99S, 1990”.

The experiment was carried out as follows:

A suspension of human PMNs (1 million cells per milliliter) was exposed to the substances to be tested (incubation for 24 h at 37° C. in an atmosphere containing 5% by volume CO₂). The number of PMN cells was determined. The PMNs were activated with 0.1 ml zymosan (an insoluble carbohydrate from the cell wall of yeast), followed by incubation for 0.5 h at 37° C. in an atmosphere containing 5% by volume CO₂).

The results are set out in the following Table (% by comparison with the control test). The quantity of ROS was measured with luminol; luminescence was determined for 60 seconds.

| | | Content of reactive oxygen compounds |
|---|---|---|
| | Dose (%) | (ROS) (% versus control) |
| Control | 0 | 100 |
| Pure engeletin | 0.001 | 65 |
| | 0.003 | 50 |
| | 0.01 | 25 |
| Pure astilbin | 0.0003 | 47 |
| | 0.001 | 26 |
| | 0.003 | 14 |

Both astilbin and engeletin are very effective in suppressing inflammatory processes. Their effectiveness increases with the quantity used.

1-9. (canceled)

10. A cosmetic, pharmaceutical or dermatological preparation comprising: from 0.001% to 5% by weight of at least one active member selected from the group consisting of extract of the plant Eperua falcata, active principles of the plant Eperua falcata, astilbin and engeletin.

11. The preparation of claim 10 wherein the active member is in the form of particles of at least one member selected from the group consisting of microcapsules, liposomes and pro-liposomes.

12. The preparation of claim 10 wherein the active member is in the form of particles with a particle size in the range of 100 to 300 nm.

13. The preparation of claim 12 wherein the particles are in the form of at least one member selected from the group consisting of microcapsules, liposomes and pro-liposomes.

14. The preparation of claim 10 wherein the preparation comprises at least one therapeutic preparation selected from the group consisting of cosmetic, pharmaceutical and dermatological preparations.

15. The preparation of claim 10 comprising a skin or hair treatment preparation.

16. A method of inhibiting neuropeptides which comprises: applying to the locus where the inhibition is required at least one member selected from the group consisting of extracts of the plant Eperua falcata, active principles of the plant Eperua falcata, astilbin and engeletin.

17. A method for reducing the release of pro-inflammatory mediators and neuropeptides of at least one of stressed skin cells and stressed neuronal fibers which comprises: applying to the locus where the reduction is required at least one member selected from the group consisting of extracts of the plant Eperua falcata, active principles of the plant Eperua falcata, astilbin and engeletin.

18. The method of claim 17 wherein the mediators or neural peptides comprise at least one of CGRP and SP.

19. A method for treating at least one member selected from the group consisting of neurogenous inflammations, sensitive skin, acne and scalp itch which comprises: applying to the locus where treatment is required at least one member selected from the group consisting of extracts of the plant Eperua falcata, active principles of the plant Eperua falcata, astilbin and engeletin.

20. The preparation of claim 10 wherein the preparation comprises an extract of the plant Eperua falcata which contains no eperua acid.

21. The preparation of claim 10 comprising a water containing extract of the plant Eperua falcata.

22. The preparation of claim 11 comprising a water containing extract of the plant Eperua falcata.

23. The preparation of claim 12 comprising a water containing extract of the plant Eperua falcata.

24. The preparation of claim 13 comprising a water containing extract of the plant Eperua falcata.

25. The preparation of claim 14 comprising a water containing extract of the plant Eperua falcata.

26. The preparation of claim 15 comprising a water containing extract of the plant Eperua falcata.

27. The method of claim 16 which comprises: applying to the locus where inhibition is required, a composition comprising a water-containing extract of the plant Eperua falcata.

28. The method of claim 17 which comprises: applying to the locus where inhibition is required, a composition comprising a water-containing extract of the plant Eperua falcata.

29. The method of claim 18 which comprises: applying to the locus where inhibition is required, a composition comprising a water-containing extract of the plant Eperua falcata.
